(19) United States
(12) Patent Application Publication
(10) Pub. No.: US 2003/0161804 A1
(43) Pub. Date: Aug. 28, 2003

(54) Self-adhesive cationic or amphoteric free-radical polymers and cosmetic use thereof

(76) Inventors: Beatrice Perron, Jouy En Josas (FR); Serge Restle, Saint Prix (FR); Nathalie Mougin, Paris (FR)

Correspondence Address: NIXON & VANDERHYE, PC, 1100 N GLEBE ROAD, 8TH FLOOR, ARLINGTON, VA, 22201-4714, US

(21) Appl. No.: 10/324,077

(22) Filed: Dec. 20, 2002

(30) Foreign Application Priority Data
Dec. 20, 2001 (FR) FR 0116597

(51) Int. Cl. A61K 7/06; A61K 7/11

(52) U.S. Cl. 424/70.11; 424/70.16; 526/277; 526/307

(57) ABSTRACT

The invention relates to cationic or amphoteric free-radical polymers, comprising one or more units derived from cationic or ampholytic ethylenic monomers, comprising at least one tertiary or quaternary amine function, characterized in that they have a self-adhesion value—expressed by the maximum tensile force (Fₘₐₓ (in N)) recorded during the detachment by traction of two circular surfaces of 0.95 cm², coated with polymer—of greater than or equal to 2 N. and also to their cosmetic use, in particular in the field of styling.

The present invention relates to novel particular self-adhesive cationic or amphoteric polymers, obtained by free-radical polymerization, to the cosmetic use of these self-adhesive polymers, and also to cosmetic compositions, and in particular styling compositions, containing them.

Water-soluble cationic polymers, such as polymers based on dimethyldiallylammonium chloride, have been used for a long time in cosmetics, and in particular in haircare. The reason for this is that their good affinity (substantivity) for keratin substrates, and in particular their capacity to form continuous films around hairs, make them excellent candidates for protecting, enhancing and strengthening the hair or alternatively to aid in depositing and fixing other substances to keratin fibres.

However, on account of their high viscosity and their incompatibility with the majority of propellants, the polymers of this family are difficult to use in aerosol products such as lacquers.

The cationic polymers commonly used in haircare moreover have low self-adhesion, i.e. hair fibres surrounded with a coat of these cationic polymers adhere very little or not at all to each other.

The Applicant has discovered a novel family of particular cationic or amphoteric free-radical polymers with a high level of self-adhesion, which have sufficient substantivity and very good styling power. This combination of properties makes them particularly suitable for use in rinse-out styling compositions such as styling shampoos.

Needless to say, their use in leave-in styling products is also advantageous since these self-adhesive cationic or amphoteric polymers may then be used in markedly smaller amounts than the known cationic or amphoteric polymers or anionic or neutral self-adhesive polymers. The possibility of using the polymers of the present invention in small amounts facilitates their formulation and reduces the viscosity of the compositions obtained.

The self-adhesive cationic or amphoteric free-radical polymers of the present invention may also be used in cosmetic fields other than that of styling. Thus, the introduction of small amounts of these polymers into the majority of makeup products ensures good adhesion of the cosmetic deposits to the skin and gives them good cohesiveness and suppleness. The makeup does not crack and does not make the users' skin taut.

One subject of the invention is, consequently, cationic or amphoteric free-radical polymers having a self-adhesion value—expressed by the maximum tensile force (Fₘₐₓ (in N)) recorded during the detachment by traction of two circular surfaces of 0.95 cm², coated with polymer—of greater than 2 N and comprising one or more units derived from monomers chosen from those of formulae (Ia), (Ib), (Ic), (Id) and (Ie)

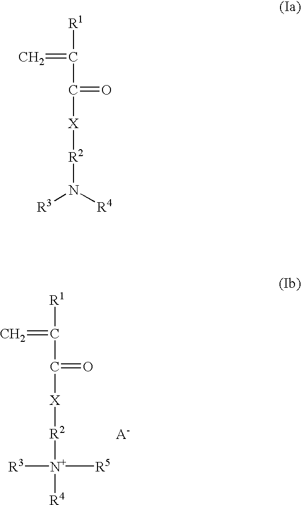

in which

R₁ represents a hydrogen atom or a methyl group,

R₂ represents a linear, branched, cycloaliphatic or aromatic C₁₋₃₀ divalent hydrocarbon-based group, which may contain one or more hetero atoms chosen from O, N and P,

R₃, R₄ and R₅ each independently represent a linear, branched, cycloaliphatic or aromatic C₁₋₃₀ hydrocarbon-based group, which may contain one or more hetero atoms chosen from O, N and P,

X represents an oxygen atom or an NH group,

A⁻ represents the counterion of the quaternary amine, preferably chosen from halide, sulphate, phosphate and carboxylate ions such as acetate,

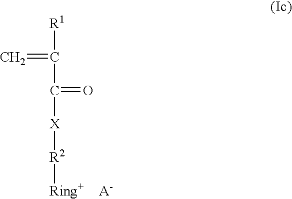

in which

X, R₁ and R₂ have the meaning given for formulae (Ia) and (Ib), and

Ring⁺ represents a monocyclic or fused bicyclic, cycloaliphatic or aromatic system, comprising a tertiary or quaternary amine function, and possibly containing one or more additional hetero atoms chosen from O, N and P;

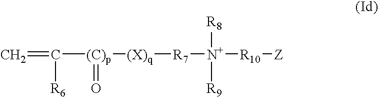

in which

R₆ represents a hydrogen atom or a linear or branched C₁₋₄ alkyl group,

R₈ and R₉ each independently represent a hydrogen atom or a linear or branched C₁₋₄ alkyl group optionally bearing a COO⁻, SO₃⁻ or PO₃H⁻ group,

R₇ and R_{1 0} each independently represent a divalent hydrocarbon-based group, in particular a group —(CH₂)ₙ with n being between 1 and 4 inclusive, optionally interrupted with an oxygen atom,

X is an oxygen atom or an NH group,

p and q are 0 or 1

Z represents a COO⁻, SO₃⁻ or PO₃H⁻ group,

in which R₇ may form with R₈, R₉ or X, when the latter represents an NH group, an aromatic or non-aromatic 5-, 6- or 7-membered heterocycle;

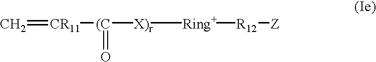

in which

R₁₁ represents a hydrogen atom or a methyl group,

R₁₂ represents a divalent C₁₋₄ hydrocarbon-based group,

X represents an oxygen atom or an NH group,

r is 0 or 1,

Ring⁺ represents a monocyclic or fused bicyclic, cycloaliphatic or aromatic system, comprising a tertiary or quaternary amine function, and possibly containing one or more additional hetero atoms chosen from O, N and P, and

Z represents a COO⁻, SO₃³¹ or PO₃H⁻ group.

A subject of the invention is also a cosmetic composition, and in particular a styling composition, containing, in a cosmetically acceptable medium, at least one such self-adhesive cationic or amphoteric free-radical polymer.

Finally, a subject of the invention is the cosmetic use of the novel self-adhesive cationic or amphoteric free-radical polymers described above, and in particular a process for treating keratin materials and a styling process using these polymers.

The self-adhesive nature of the cationic or amphoteric polymers of the present invention is assessed according to the following protocol.

40 μl of an aqueous solution or dispersion containing 10% by weight of test polymer are deposited on the surface of two circular frosted-glass plates, each having a surface area of 0.95 cm² (11 mm diameter). The plates are left to dry for 48 hours at ambient pressure, at a relative humidity of 55% and at a temperature of 22° C.

The two plates are mounted in a machine for measuring tensile strength (Lloyd LR5K) and are pressed together for 20 seconds with a force of 3 N. The two plates are then separated, under the same temperature and relative humidity conditions, for 30 seconds by imposing a traction speed of 20 mm/minute, and the force required for this displacement, and more particularly the maximum force (Fₘₐₓ) in newtons (N) measured at the time of the sudden separation of the two polymer-coated surfaces, is recorded. Obviously, the self-adhesion of the polymers of the present invention is proportionately greater the larger the maximum force recorded.

The cationic or amphoteric self-adhesive free-radical polymers of the present invention have a self-adhesion value such that Fₘₐₓ is greater than or equal to 2 N, preferably between 2 and 100 and in particular between 5 N and 100 N.

The self-adhesive cationic or amphoteric free-radical polymers of the present invention preferably have a glass transition temperature (Tg) of less than room temperature (20° C.), i.e. at room temperature, they are in plastic form rather than in vitreous form. The self-adhesion characteristics of the polymers of the present invention are particularly advantageous when the glass transition temperature is less than 0° C. and in particular less than −20° C.

When the cationic or amphoteric free-radical polymers of the present invention have several glass transition temperatures, the lowest Tg is preferably less than 20° C., in particular less than 0° C. and ideally less than −20° C.

The glass transition temperature of the polymers of the present invention is measured by Differential Scanning Calorimetry (DSC) under the following conditions:

To measure the glass transition temperature, a film about 150 mm thick of test polymer is prepared by depositing an aqueous solution or dispersion of the polymer in a circular Teflon die 40 mm in diameter and leaving the deposit to dry. The film is dried in an oven at a temperature of about 23° C. under a relative humidity of 45%, until the weight no longer changes. About 5 to 15 mg of the film are taken up and placed in a crucible, which is then introduced into the analyser. The thermal analyser is a DSC-2920 model from the company TA Instruments. The initial and final temperatures of the temperature sweep are chosen so as to surround the desired glass transition temperature. The temperature sweep is performed at a rate of 10° C./minute.

This analysis is performed according to ASTM standard D 3418-97 apart from the above changes.

Examples of cationic ethylenic monomers defined by formulae (Ia) to (Ic) above that may be mentioned include dimethylaminoethyl (meth)acrylate, diethylaminoethyl (meth)acrylate, dimethylaminopropyl (meth)acrylate, dimethylaminoethyl(meth)acrylamide, dimethylaminopropyl(meth)acrylamide, N-morpholinoethyl (meth)acrylate, trimethylammonioethyl (meth)acrylate chloride, trimethylammoniopropyl (meth)acrylate chloride, trimethylammonioethyl(meth)acrylamide chloride, trimethylammoniopropyl(meth)acrylamide chloride and dimethylbenzylammonioethyl (meth)acrylate chloride.

As examples of amphoteric monomers of formula (Id) or (Ie) that are particularly preferred, mention may be made of 1-vinyl-2-(3-sulphopropyl)imidazolium hydroxide, 1-vinyl-3-(3-sulphopropyl)imidazolium, 1-vinyl-3-(4-sulphobutyl)imidazolium hydr-oxide, 1-vinyl-2-methyl-3-(4-sulphobutyl)imidazolium hydroxide, 2-vinyl-1-(3-sulphopropyl)pyridinium hydr-oxide, 2-methyl-5-vinyl-1-(3-sulphopropyl)pyridinium hydroxide, 4-vinyl-1-(3-sulphopropyl)pyridinium hydr-oxide, dimethyl(2-methacryloxyethyl) (3-sulphopropyl)ammonium hydroxide, diethyl(2-methacryloxyethoxy)-2-ethyl(3-sulphopropyl)-ammonium hydroxide, 4-vinyl-4-(sulphobutyl)pyridinium, N-(3-sulphopropyl)-N-methacrylamidopropyl-N,N-dimethylammonium hydroxide, N,N-dimethyl-N-(3-(methacrylamido)propyl)(3-sulphopro-pyl)ammonium hydroxide, N,N-dimethyl-N-(3-methacrylamidopropyl)-N-(3-carboxypropyl)ammonium hydroxide and N,N-dimethyl-N-(2-methacryloxyethyl)-N-(3-carboxypropyl)ammonium hydroxide.

Cationic comonomers that are most particularly preferred according to the invention are dimethylaminoethyl methacrylate (DMAEMA) and dimethylaminopropylmethacrylamide (DMAPMA).

The self-adhesive cationic or amphoteric polymers of the present invention preferably comprise—besides the units derived from cationic and/or amphoteric monomers—units derived from nonionic ethylenic monomers.

The Applicant has found that the self-adhesion properties of the polymers of the present invention are particularly advantageous when these nonionic monomers are chosen from those that form, when they are homopolymerized, polymers with a glass transition temperature of less than 0° C.

These nonionic comonomers are known in the art and may be described by formula (IIa) or (IIb):

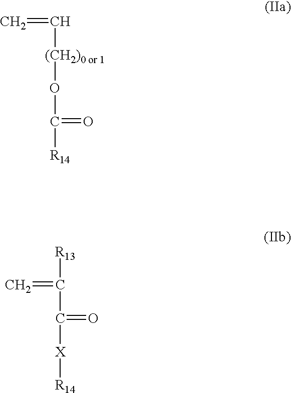

in which

R₁₃ represents a hydrogen atom or a methyl group,

X represents an oxygen atom or an NH group, and

R₁₄ represents a linear, branched, cycloaliphatic or aromatic C₂₋₆₀ hydrocarbon-based group, which may contain one or more atoms chosen from O, S and P, and especially an alkoxy-polyethylene glycol chain.

Among these nonionic comonomers optionally giving homopolymers with a Tg<0° C., the ones that are particularly preferred are ethyl, n-butyl, n-hexyl, 2-ethylhexyl, n-nonyl, lauryl, n-octadecyl, isooctyl, isodecyl, hydroxyethyl, hydroxypropyl and methoxyethyl acrylate, n-hexyl, 2-ethylhexyl, ethoxyethyl, isodecyl, methoxyethyl or C₁₋₃₀ alkoxy-PEG (with 5 to 30 ethylene oxide units) methacrylate, vinyl propionate, and vinyl neoalkanoates such as vinyl neononanoate and vinyl neododecanoate.

Comonomers of this group that are particularly preferred include n-butyl acrylate, ethoxyethyl methacrylate, 2-ethylhexyl acrylate and methoxy-PEG methacrylate (with 5 to 30 ethylene oxide units).

The self-adhesive polymers of the present invention may also contain a small amount of highly hydrophobic nonionic monomers such as vinyl monomers containing a silicone side chain, chlorotrifluoroethylene, tetrafluoroethylene, and vinyl, allylic or (meth)acrylic monomers containing a perhalohydrocarbon and in particular a perfluorohydrocarbon side chain, such as perfluorohexyl (meth)acrylate or perfluorooctyl (meth)acrylate.

When the self-adhesive cationic or amphoteric polymers of the present invention consist both of cationic or amphoteric monomers and of nonionic monomers giving homopolymers with a Tg<0° C., the units derived from cationic or amphoteric ethylenic monomers represent from 1% to 50% by weight of the polymer and preferably from 1% to 20% by weight of polymer, and the units derived from nonionic ethylenic monomers giving homopolymers with a Tg<0° C. represent from 50% to 99% by weight of the polymer and preferably from 80% to 99% by weight of the polymer.

The self-adhesive polymers of the present invention may comprise—in addition to units derived from cationic or amphoteric monomers and units derived from nonionic monomers giving homopolymers with a Tg<0° C.—a certain proportion of anionic ethylenic comonomers.

These comonomers are ethylenic monomers bearing at least one carboxylic acid, sulphonic acid or phosphonic acid function.

They are chosen, for example, from acrylic acid, methacrylic acid, itaconic acid, crotonic acid, fumaric acid, maleic acid, vinylbenzoic acid, vinylbenzenesulphonic acid, acrylamidopropanesulphonic acid and vinylphosphonic acid, or from the addition salts with mineral or organic bases of these acids.

Introducing these anionic comonomers makes it possible to adjust the equilibrium of the charges, to modify the hydrophilic nature and thus the solubility of the polymers obtained, or to modify the compatibility of the polymers with certain cosmetic substrates or supports.

To ensure good affinity of the self-adhesive polymers of the present invention with cosmetic substrates, and in particular keratin fibres, it is generally desirable that the overall charge of the amphoteric polymers should be positive, i.e. that the total number of positive charges borne by the polymers should be greater than the number of negative charges.

Among the cationic or amphoteric free-radical copolymers described above, the Applicant has obtained particularly advantageous results with the following polymers:

copolymers of butyl acrylate and of dimethylaminoethyl methacrylate,

copolymers of butyl acrylate and of dimethylaminopropylmethacrylamide,

terpolymers of methoxy-PEG methacrylate (with 5 to 30 EO units), of dimethylaminopropylmethacrylamide and of ethoxyethyl methacrylate, and

terpolymers of 2-ethylhexyl acrylate, of dimethylaminopropylmethacrylamide and of ethoxyethyl methacrylate.

Although the molecular mass of the self-adhesive cationic or amphoteric polymers of the present invention is not a deciding factor for the present invention, polymers with a number-average molar mass of between 5 000 and 5 000 000 and preferably between 50 000 and 5 000 000 are preferably used.

The self-adhesive cationic or amphoteric polymers of the present invention may be water-soluble or water-insoluble. When they are water-insoluble, they are usually in the form of latices, i.e. in the form of dispersions of fine particles, with a mean size of between 3 nm and 600 nm and preferably between 5 nm and 400 nm, in an aqueous phase.

The water-insoluble dispersed forms of the polymers of the present invention are preferred since they do not have the high-viscosity problems of solutions and are easier to handle than solutions.

The self-adhesive cationic or amphoteric polymers of the present invention may be prepared by free-radical solution, bulk, dispersion or emulsion polymerization, which are familiar to those skilled in the art.

The positive charges of the polymers may be introduced by copolymerization of monomers containing a protonated tertiary amine function or containing a quaternary amine function, but the protonation or quaternization of the amine functions may also take place after polymerization.

The agents for protonating the amine functions of the self-adhesive polymers of the present invention are chosen from cosmetically acceptable organic and mineral acids such as hydrochloric acid, acetic acid, glycolic acid and succinic acid.

Alkylating agents are known compounds containing one or more alkyl chains and a suitable leaving group such as a halogen atom or a sulphate group. Examples that may be mentioned include C₁-C₃₀ alkyl halides such as methyl chloride, and dialkyl sulphates, for instance diethyl sulphate.

As mentioned above, the self-adhesive cationic or amphoteric polymers of the present invention may be used in cosmetics in the form of care or makeup compositions for the skin or the integuments, in particular in the form of care, makeup or fixing compositions for human keratin materials such as the hair and the eyelashes.

These cosmetic compositions preferably contain, in a cosmetically acceptable aqueous medium, from 0.01% to 40%, in particular from 0.05% to 20% and ideally from 0.1% to 10% by weight, of at least one self-adhesive cationic or amphoteric polymer of the present invention.

In one preferred embodiment of the present invention, the cosmetic compositions are styling compositions, and in particular rinse-out styling compositions, i.e. styling shampoos.

The cosmetically acceptable aqueous medium may contain various adjuvants and solvents commonly used in cosmetics, such as surfactants, anionic, amphoteric, zwitterionic or nonionic polymers, cationic polymers other than the cationic self-adhesive polymers of the present invention, nacreous agents and/or opacifiers, organic solvents, fragrances, mineral, plant and/or synthetic oils, fatty acid esters, pigments and colorants, silicones, mineral or organic particles, pH stabilizers, preserving agents and UV absorbers.

Among the cationic polymers that may be used in the compositions of the present invention, the ones that are preferred are quaternary cellulose ether derivatives such as the products sold under the name “JR 400” by the company Union Carbide Corporation, cyclopolymers, in particular homopolymers of diallyldimethylammonium salt and copolymers of diallyldimethylammonium salt and of acrylamide, in particular the chlorides, sold under the names “Merquat 100”, “Merquat 550” and “Merquat S” by the company Merck, cationic polysaccharides and more particularly guar gums modified with 2,3-epoxypropyltrimethylammonium chloride, sold, for example, under the name “Jaguar C13S” by the company Meyhall, optionally crosslinked homopolymers and copolymers of (meth)acryloyloxyethyltrimethylammonium salt, sold by the company Allied Colloids as a 50% solution in mineral oil, under the trade names Salcare SC92 (crosslinked copolymer of methacryloyloxyethyltrimethylammonium chloride and of acrylamide) and Salcare SC95 (crosslinked homopolymer of methacryloyloxyethyltrimethylammonium chloride).

Polymers consisting of repeating units corresponding to the formula:

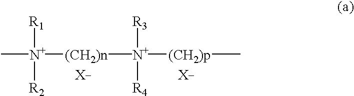

in which R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from a mineral or organic acid, may also be used.

The surfactants that may be used in the composition according to the present invention may be anionic, nonionic, amphoteric or cationic surfactants, or mixtures thereof.

Among the anionic surfactants that may be used, alone or as mixtures, in the context of the present invention, mention may be made especially of salts, and in particular alkali metal salts such as sodium salts, ammonium salts, amine salts, amino alcohol salts or alkaline-earth metal salts, for example magnesium salts, of the following compounds: alkyl sulphates, alkyl ether sulphates, alkylamido ether sulphates, alkylarylpolyether sulphates, monoglyceride sulphates; alkylsulphonates, alkylamidesulphonates, alkylarylsulphonates, α-olefin sulphonates, paraffin sulphonates; alkylsulphosuccinates, alkyl ether sulphosuccinates, alkylamide sulphosuccinates; alkylsulphoacetates; acylsarcosinates; and acylglutamates, the alkyl and acyl groups of all these compounds containing from 6 to 24 carbon atoms and the aryl group preferably denoting a phenyl or benzyl group.

In the context of the present invention, it is also possible to use C₆-C₂₄ alkyl esters of polyglycoside carboxylic acids such as alkyl glucoside citrates, polyalkyl glycoside tartrates, and polyalkyl glycoside sulphosuccinates; alkylsulphosuccinimates, acylisethionates and N-acyltaurates, the alkyl or acyl group of all these compounds containing from 12 to 20 carbon atoms. Among the anionic surfactants that may also be used, mention may also be made of acyllactylates in which the acyl group contains from 8 to 20 carbon atoms.

In addition, mention may also be made of alkyl-D-galactosideuronic acids and the salts thereof, and also polyoxyalkylenated (C₆-C₂₄)alkyl ether carboxylic acids, polyoxyalkylenated (C₆-C2₄)alkyl(C6-C₂₄)aryl ether carboxylic acids, polyoxyalkylenated (C₆-C₂₄)alkylamido ether carboxylic acids and salts thereof, in particular those containing from 2 to 50 ethylene oxide groups, and mixtures thereof.

Among the abovementioned anionic surfactants that are preferably used according to the invention are (C₆-C₂₄) alkyl sulphates, (C₆-C₂₄) alkyl ether sulphates, (C₆-C₂₄)alkyl ether carboxylates and mixtures thereof, for example ammonium lauryl sulphate, sodium lauryl sulphate, magnesium lauryl sulphate, sodium lauryl ether sulphate, ammonium lauryl ether sulphate and magnesium lauryl ether sulphate.

The composition according to the present invention may comprise the anionic surfactants in an amount preferably of between 0.5% and 60% by weight and better still between 5% and 20% by weight, relative to the total weight of the composition.

The nonionic surfactants that may be used in the context of the present invention are, themselves also, compounds that are well known per se (see in particular in this respect “Handbook of Surfactants” by M. R. Porter, published by Blackie & Son (Glasgow and London), 1991, pp. 116-178). They can be chosen in particular from (non-limiting list) polyethoxylated, polypropoxylated or polyglycerolated fatty acids, alkylphenols, α-diols or alcohols having a fatty chain containing, for example, 8 to 18 carbon atoms, it being possible for the number of ethylene oxide or propylene oxide groups to range in particular from 2 to 50 and for the number of glycerol groups to range in particular from 2 to 30. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; polyethoxylated fatty amines preferably having 2 to 30 mol of ethylene oxide; oxyethylenated fatty acid esters of sorbitan having from 2 to 30 mol of ethylene oxide; fatty acid esters of sucrose, fatty acid esters of polyethylene glycol, (C₆-C₂₄)alkylpolyglucosides, N-(C₆-C₂₄)alkylglucamine derivatives, amine oxides such as (C₁₀-C₁₄)alkylamine oxides or N-(C₁₀-C₁₄)acylaminopropylmorpholine oxides; and mixtures thereof.

Among the abovementioned nonionic surfactants that are preferably used are (C₆-C₂₄)alkylpolyglycosides, in particular decylpolyglucoside.

The amphoteric surfactants that are suitable for use in the present invention may especially be aliphatic secondary or tertiary amine derivatives, in which the aliphatic group is a linear or branched chain containing 8 to 22 carbon atoms and containing at least one water-soluble anionic group, such as, for example, a carboxylate, sulphonate, sulphate, phosphate or phosphonate group; mention may also be made of (C₈-C₂₀)alkylbetaines, sulphobetaines, (C₈-C₂₀)alkylamido(C₆-C₈)alkylbetaines or (C₈-C₂₀)alkylamido(C₆-C₈)-alkylsulphobetaines; and mixtures thereof.

Among the amine derivatives that may be mentioned are the products sold under the name Miranol®, as described in U.S. Pat. Nos. 2,528,378 and 2,781,354 and classified in the CTFA dictionary, 3rd edition, 1982, under the names Amphocarboxyglycinate and amphocarboxypropionate, and having the respective structures (1) and (2):

R₂—CONHCH₂CH₂—N⁺(R₃)(R₄)(CH₂COO⁻) (1)

in which:

R₂ represents an alkyl group derived from an acid R₂—COOH present in hydrolysed coconut oil, or a heptyl, nonyl or undecyl group,

R₃ represents a β-hydroxyethyl group, and

R₄ represents a carboxymethyl group; and

R₂′—CONHCH₂CH₂—N(B)(C) (2)

in which:

B represents —CH₂CH₂OX′,

C represents —(CH₂)_{z}—Y′, with z=1 or 2,

X′ represents the —CH₂CH₂—COOH group or a hydrogen atom,

Y′ represents —COOH or the —CH₂-CHOH-SO₃H group,

R₂′ represents the alkyl group of an acid R₂′—COOH present in coconut oil or in hydrolysed linseed oil, an alkyl group, especially a C₁₇ group and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, mention may be made of the cocoamphodiacetate sold under the trade name Miranol® C2M concentrate by the company Rhodia.

Among the amphoteric surfactants that are preferably used are (C8-C₂₀)alkylbetaines such as cocobetaine, (C₈-C₂₀)alkylamido(C₆-C₈)alkylbetaines such as cocamidobetaine, and alkylamphodiacetates, for instance disodium cocoamphodiacetate, and mixtures thereof.

The composition according to the invention may also comprise one or more cationic surfactants that are well known per se, such as primary, secondary or tertiary fatty amine salts, optionally polyoxyalkylenated; quaternary ammonium salts such as tetraalkylammonium, alkylamidoalkyltrialkylammonium, trialkylbenzylammonium, trialkylhydroxyalkylammonium or alkylpyridinium chlorides or bromides; imidazoline derivatives; or amine oxides of cationic nature.

The nonionic, amphoteric and cationic surfactants described above may be used alone or as mixtures and the amount thereof is between 0.1% and 30% by weight, preferably between 0.5% and 25% by weight and better still between 1% and 20% by weight, relative to the total weight of the composition.

The silicones that may be used as additives in the cosmetic compositions of the present invention are volatile or non-volatile, cyclic, linear or branched silicones, optionally modified with organic groups, having a viscosity from 5×10⁶ to 2.5 m²/s at 25° C. and preferably 1×10⁵ to 1 m²/s.

The silicones that may be used in accordance with the invention may be soluble or insoluble in the composition and in particular may be polyorganosiloxanes that are insoluble in the composition of the invention. They may be in the form of oils, waxes, resins or gums.

The organopolysiloxanes are defined in greater detail in Walter Noll's “Chemistry and Technology of Silicones” (1968), Academic Press. They can be volatile or non-volatile.

When they are volatile, the silicones are more particularly chosen from those having a boiling point of between 60° C. and 260° C., and even more particularly from:

(i) cyclic silicones containing from 3 to 7 and preferably 4 to 5 silicon atoms. These are, for example, octamethylcyclotetrasiloxane sold in particular under the name Volatile Silicone® 7207 by Union Carbide or Silbione® 70045 V 2 by Rhodia, decamethylcyclopentasiloxane sold under the name Volatile Silicone® 7158 by Union Carbide, and Silbione® 70045 V 5 by Rhodia, and mixtures thereof.

Mention may also be made of cyclocopolymers of the dimethylsiloxane/methylalkylsiloxane type, such as Silicone Volatile® FZ 3109 sold by the company Union Carbide, of formula:

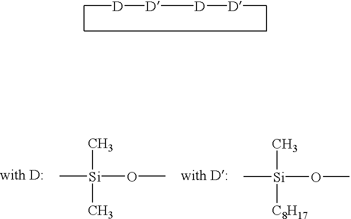

Mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Non-volatile silicones, and more particularly polyalkylsiloxanes, polyarylsiloxanes, polyalkylarylsiloxanes, silicone gums and resins, polyorganosiloxanes modified with organofunctional groups, and mixtures thereof, are preferably used.

These silicones are more particularly chosen from polyalkylsiloxanes, among which mention may be made mainly of polydimethylsiloxanes containing trimethylsilyl end groups. The viscosity of the silicones is measured at 25° C. according to ASTM standard 445 Appendix C.

Among these polyalkylsiloxanes, mention may be made, in a non-limiting manner, of the following commercial products:

the Silbione® oils of the 47 and 70 047 series or the Mirasil® oils sold by Rhodia, such as, for example, the oil 70 047 V 500 000;

the oils of the Mirasil® series sold by the company Rhodia;

the oils of the 200 series from the company Dow Corning, such as, DC200 with a viscosity of 60 000 mm²/s;

the Viscasil® oils from General Electric and certain oils of the SF series (SF 96, SF 18) from General Electric.

Mention may also be made of polydimethylsiloxanes containing dimethylsilanol end groups, known by the name dimethiconol (CTFA), such as the oils of the 48 series from the company Rhodia.

In this category of polyalkylsiloxanes, mention may also be made of the products sold under the names Abil Wax® 9800 and 9801 by the company Goldschmidt, which are poly(C₁-C₂₀)alkylsiloxanes.

The polyalkylarylsiloxanes are chosen particularly from linear and/or branched polydimethyl/methylphenylsiloxanes and polydimethyldiphenylsiloxanes with a viscosity of from 1×10⁻⁵ to 5×10⁻² m²/s at 25° C.

Among these polyalkylarylsiloxanes, mention may be made, by way of example, of the products sold under the following names:

the Silbione® oils of the 70 641 series from Rhodia;

the oils of the Rhodorsil® 70 633 and 763 series from Rhodia;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000;

certain oils of the SF series from General Electric, such as SF 1023, SF 1154, SF 1250 and SF 1265.

The silicone gums that can be used in accordance with the invention are, in particular, polydiorganosiloxanes having high number-average molecular masses of between 200 000 and 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane and tridecane, or mixtures thereof.

Mention may be made more particularly of the following products:

polydimethylsiloxane

polydimethylsiloxane/methylvinylsiloxane gums,

polydimethylsiloxane/diphenylsiloxane,

polydimethylsiloxane/phenylmethylsiloxane,

polydimethylsiloxane/diphenylsiloxane/methylvinylsiloxane.

Products that can be used more particularly in accordance with the invention are mixtures such as:

mixtures formed from a polydimethylsiloxane hydroxylated at the end of the chain, or dimethiconol (CTFA) and from a cyclic polydimethylsiloxane also called cyclomethicone (CTFA), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;

mixtures of two PDMSs of different viscosities, and more particularly of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product preferably contains 15% SE 30 gum and 85% SF 96 oil.

The organopolysiloxane resins that can be used in accordance with the invention are crosslinked siloxane systems containing the following units:

R₂SiO_{2/2}, R₃SiO_{1/2}, RSiO_{3/2} and SiO_{4/2} in which R represents a hydrocarbon-based group containing 1 to 16 carbon atoms or a phenyl group. Among these products, those particularly preferred are the ones in which R denotes a C₁-C₄ lower alkyl group, more particularly methyl, or a phenyl group.

Among these resins, mention may be made of the product sold under the name Dow Corning 593 or those sold under the names Silicone Fluid SS 4230 and SS 4267 by the company General Electric, which are silicones of dimethyl/trimethylsiloxane structure.

Mention may also be made of the trimethyl siloxysilicate type resins sold in particular under the names X22-4914, X21-5034 and X21-5037 by the company Shin-Etsu.

The organomodified silicones that can be used in accordance with the invention are silicones as defined above and containing in their structure one or more organofunctional groups attached via a hydrocarbon-based group.

Among the organomodified silicones, mention may be made of polyorganosiloxanes comprising:

polyethyleneoxy and/or polypropyleneoxy groups optionally containing C₆-C₂₄ alkyl groups, such as the products known as dimethicone copolyol sold by the company Dow Corning under the name DC 1248 or the oils Silwet® L 722, L 7500, L 77, L 711 from the company Union carbide and the (C₁₂)alkylmethicone copolyol sold by the company Dow Corning under the name Q2 5200;

substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C₁-C₄ aminoalkyl groups;

thiol groups such as the products sold under the names GP 72 A and GP 71 from Genesee;

alkoxylated groups such as the product sold under the name Silicone Copolymer F-755 by SWS Silicones and Abil Wax® 2428, 2434 and 2440 by the company Goldschmidt;

hydroxylated groups such as the polyorganosiloxanes containing a hydroxyalkyl function, described in French patent application FR-A-85/16334;

acyloxyalkyl groups such as, for example, the polyorganosiloxanes described in U.S. Pat. No. 4,957,732;

anionic groups of carboxylic type, such as, for example, in the products described in patent EP 186 507 from the company Chisso Corporation, or of alkylcarboxylic type, such as those present in the product X-22-3701E from the company Shin-Etsu; 2-hydroxyalkyl sulphonate; 2-hydroxyalkyl thiosulphate such as the products sold by the company Goldschmidt under the names Abil® S201 and Abil® S255;

hydroxyacylamino groups, such as the polyorganosiloxanes described in patent application EP 342 834. Mention may be made, for example, of the product Q2-8413 from the company Dow Corning.

The silicones as described above may be used, alone or as a mixture, in an amount of between 0.01% and 20% by weight and preferably between 0.1% and 5% by weight.

The cosmetically acceptable aqueous medium may contain mineral or organic electrolytes.

The electrolytes used are preferably water-soluble mineral salts such as alkali metal, alkaline-earth metal or aluminium salts, hydrochloric acid, sulphuric acid or nitric acid salts, or alternatively organic acid salts such as alkali metal, alkaline-earth metal or aluminium carbonates, lactates, citrates or tartrates. The electrolytes that are particularly preferred are chosen from potassium sulphate, sodium sulphate, magnesium sulphate, calcium nitrate, magnesium nitrate, sodium chloride, potassium chloride, potassium carbonate, sodium carbonate and sodium citrate.

These electrolytes are preferably present in proportions ranging from 0.1% to 30% by weight and in particular from 1% to 10% by weight, relative to the total weight of the composition.

The pH of the aqueous compositions of the present invention is preferably set at a value of between 3 and 11 and in particular between 5 and 9.

A subject of the present invention is also a process for treating keratin materials, comprising the application of a cosmetic composition according to the present invention to the keratin materials to be treated.

A subject of the invention is also a styling process comprising the application of a cosmetic composition according to the present invention to the hair, rinsing the hair, and then shaping and drying the rinsed hair.

The invention is illustrated with the aid of the following examples, which constitute preferred embodiments of the polymers and compositions of the present invention.

### EXAMPLE 1

85 g of butyl acrylate and 15 g of dimethylaminoethyl methacrylate (DMAEMA) are introduced into a reactor equipped with a stirring system, a condenser and a thermometer. The mixture is diluted with 100 g of tetrahydrofuran and 1 g of initiator (Trigonox® 21, sold by the company Akzo) is added. The resulting mixture is heated at the reflux point of the solvent for 6 hours.

GC chromatographic analysis of the polymer thus obtained is carried out in tetrahydrofuran (polystyrene calibration) on a sample of polymer purified by precipitation from water. The mass at the maximum elution peak is equal to 80 000.

200 g of THF are added to the above solution, followed by addition of 1.06 g of HCl dissolved in 100 g of water, while maintaining vigorous stirring using an Ultra-Turrax machine. This amount of hydrochloric acid corresponds to the amount required to neutralize one third of the units derived from dimethylaminoethyl methacrylate, i.e. 0.029 mol per 100 g of polymer. After this step of partial neutralization, the tetrahydrofuran is evaporated off so as to obtain an aqueous dispersion of the polymer having a concentration of 20%. The size of the particles obtained, determined by light scattering using a Coulter N4SD machine, is 213 nm.

The maximum tensile force (Fₘₐₓ (in N)) recorded during detachment by traction of two circular surfaces of 0.95 cm², coated with this copolymer of butyl acrylate and of dimethylaminoethyl methacrylate, is 6.0 N.

### EXAMPLE 2

90 g of butyl acrylate and 10 q of dimethylaminopropylmethacrylamide (DMAPMA) are introduced into a reactor equipped with a stirring system, a condenser and a thermometer. The mixture is diluted with 100 g of tetrahydrofuran and 1 g of initiator (Trigonox® 21, sold by the company Akzo) is added. The resulting mixture is heated at the reflux point of the solvent for 6 hours.

GC chromatographic analysis of the polymer thus obtained is carried out in tetrahydrofuran (polystyrene calibration) on a sample of polymer purified by precipitation from water. The mass at the maximum elution peak is equal to 80 000.

200 g of THF are added to the above solution, followed by addition of 2.33 g of HCl dissolved in 100 g of water, while maintaining vigorous stirring using an Ultra-Turrax machine. This amount of hydrochloric acid corresponds to the amount required to neutralize 100% of the units derived from DMAPMA, i.e. 0.064 mol per 100 g of polymer. After this step of neutralization, the tetrahydrofuran is evaporated off so as to obtain an aqueous dispersion of the polymer having a concentration of 20%. The average size of the particles obtained, determined by light scattering using a Coulter N4SD machine, is 18.6 nm.

The maximum tensile force (Fₘₐₓ (in N)) recorded during detachment by traction of two circular surfaces of 0.95 cm², coated with this copolymer of butyl acrylate and dimethylaminopropylmethacrylamide is, 11.4 N.

### EXAMPLE 3

The following shampoos A and B and conditioner C are prepared:

| | Shampoo | Shampoo | Conditioner |
|---|---|---|---|
| | A | B | C |
| Sodium lauryl ether | 12.5% of | 12.5% of | |
| sulphate (2.2 EO) | active | active | |
| containing 70% active | material | material | |
| material | | | |
| Cocoylbetaine as an | 2.5% of | 2.5% of | |
| aqueous 30% solution | active | active | |
| | material | material | |
| Mixture of cetylstearyl | | | 4% of |
| alcohol and of | | | active |
| oxyethylenated (30 EO) | | | material |
| cetylstearyl alcohol | | | |
| Behenyltrimethylammonium | | | 2% of |
| chloride at 80% in a | | | active |
| water/isopropanol mixture | | | material |
| (15/85) | | | |
| Polymer according to | 3% of | | |
| Example 1 | active | | |
| | material | | |
| Polymer according to | | 3% of | 3% of |
| Example 2 | | active | active |
| | | material | material |
| Water | qs 100% | qs 100% | qs 100% |

Hair washed or treated with these compositions has advantageous cosmetic properties in terms of amount of body and ease of styling.

### EXAMPLE 4

A cationic terpolymer is prepared in a similar manner to that described in Examples 1 and 2, starting with 40 g of methoxypolyethylene glycol methacrylate (molecular mass 550), 50 g of dimethylaminopropylmethacrylamide and 10 g of ethoxyethyl methacrylate in 100 g of tetrahydrofuran in the presence of 1 g of initiator (Trigonox® 21, sold by the company Akzo).

### EXAMPLE 5

A cationic terpolymer is prepared in a manner similar to that described in Examples 1 and 2, starting with 80 g of 2-ethylhexyl acrylate, 10 g of dimethylaminopropylmethacrylamide (DMAPMA) and 10 g of ethoxyethyl methacrylate in 100 g of tetrahydrofuran in the presence of 1 g of initiator (Trigonox® 21, sold by the company Akzo).

### EXAMPLE 6

The following shampoos D and E are prepared:

| | Shampoo D | Shampoo E |
|---|---|---|
| Sodium lauryl ether | 12.5% of active | 12.5% of active |
| sulphate (2.2 EO) at | material | material |
| 70% active material | | |
| Cocoylbetaine as an | 2.5% of active | 2.5% of active |
| aqueous 30% solution | material | material |
| Merquat ® 100 (Nalco) | 0.1% of active | 0.1% of active |
| | material | material |
| Terpolymer according | 3% of active | |
| to Example 4 | material | |
| Terpolymer according | | 3% of active |
| to Example 5 | | material |
| Water | qs 100% | qs 100% |

Hair washed or treated with these shampoos has advantageous cosmetic properties in terms of amount of body and ease of styling.

1. Cationic or amphoteric free-radical polymers, comprising one or more units derived from cationic or amphoteric ethylenic monomers, comprising at least one tertiary or quaternary amine function, characterized in that they have a self-adhesion value, expressed by the maximum tensile force (Fₘₐₓ (in N)), of greater than or equal to 2 N, and in that they comprise one or more units derived from monomers chosen from those of formulae (Ia), (Ib), (Ic), (Id) and (Ie) 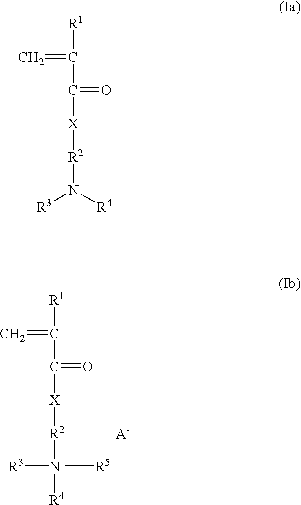
in which
R₁ represents a hydrogen atom or a methyl group,
R₂ represents a linear, branched, cycloaliphatic or aromatic C₁₋₃₀ divalent hydrocarbon-based group, which may contain one or more hetero atoms chosen from O, N and P,
R₃, R₄ and R₅ each independently represent a linear, branched, cycloaliphatic or aromatic C₁₋₃₀ hydrocarbon-based group, which may contain one or more hetero atoms chosen from O, N and P,
X represents an oxygen atom or an NH group,
A⁻ represents the counterion of the quaternary amine, preferably chosen from halide, sulphate, phosphate and carboxylate ions such as acetate, 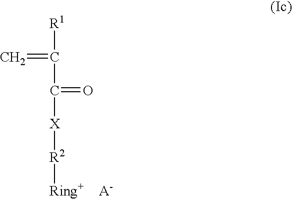
in which
X, R₁ and R₂ have the meaning given for formulae (Ia) and (Ib), and
Ring⁺ represents a monocyclic or fused bicyclic, cycloaliphatic or aromatic system, comprising a tertiary or quaternary amine function, and possibly containing one or more additional hetero atoms chosen from O, N and P; 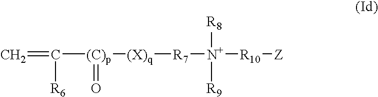
in which
R₆ represents a hydrogen atom or a linear or branched C₁₋₄ alkyl group,
R₈ and R₉ each independently represent a hydrogen atom or a linear or branched C₁₋₄ alkyl group optionally bearing a COO⁻, SO₃⁻ or PO₃H⁻ group,
R₇ and R₁₀ each independently represent a divalent hydrocarbon-based group, in particular a group —(CH₂)n with n being between 1 and 4 inclusive, optionally interrupted with an oxygen atom,
X is an oxygen atom or an NH group,
p and q are 0 or 1
Z represents a COO⁻, SO₃⁻ or PO₃H⁻ group,
in which R₇ may form with R₈, R₉ or X, when the latter represents an NH group, an aromatic or non-aromatic 5-, 6- or 7-membered heterocycle; 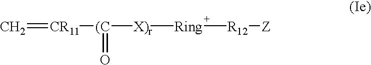
in which
R₁₁ represents a hydrogen atom or a methyl group,
R₁₂ represents a divalent C₁₋₄ hydrocarbon-based group,
X represents an oxygen atom or an NH group,
r is 0 or 1,
Ring⁺ represents a monocyclic or fused bicyclic, cycloaliphatic or aromatic system, comprising a tertiary or quaternary amine function, and possibly containing one or more additional hetero atoms chosen from O, N and P, and
Z represents a COO⁻, SO₃⁻ or PO₃H⁻ group.

2. Polymers according to claim 1, characterized in that Fₘₐₓ is between 2 N and 100 N and preferably between 5 and 100 N.

3. Polymers according to claim 1 or 2, characterized in that they have a glass transition temperature (Tg), determined by differential scanning calorimetry, of less than 20° C., preferably less than 0° C. and in particular less than −20° C.

4. Polymers according to any one of the preceding claims, characterized in that the cationic ethylenic monomers of formulae (Ia) to (Ic) comprising at least one tertiary amine function are chosen from dimethylaminoethyl (meth)acrylate, diethylaminoethyl (meth)acrylate, dimethylaminopropyl (meth)acrylate, dimethylaminoethyl(meth)acrylamide, dimethylamino-propyl(meth)acrylamide, N-morpholinoethyl (meth)-acrylate, trimethylammonioethyl (meth)acrylate chloride, trimethylammoniopropyl (meth)acrylate chloride, trimethylammonioethyl(meth)acrylamide chloride, trimethylammoniopropyl(meth)acrylamide chloride and dimethylbenzylammonioethyl (meth) acrylate chloride.

5. Polymers according to claim 4, characterized in that the cationic ethylenic monomers are chosen from dimethylaminoethyl methacrylate (DMAEMA) and dimethylaminopropylmethacrylamide (DMAPMA).

6. Polymers according to any one of the preceding claims, characterized in that the amphoteric monomers of formulae (Id) and (le) are chosen from 1-vinyl-2-(3-sulphopropyl)imidazolium hydroxide, 1-vinyl-3-(3-sulphopropyl)imidazolium, 1-vinyl-3-(4-sulphobutyl)imidazolium hydroxide, 1-vinyl-2-methyl-3-(4-sulphobutyl)imidazoiium hydroxide, 2-vinyl-1-(3-sulphopropyl)pyridinium hydroxide, 2-methyl-5-vinyl-1-(3-sulphopropyl)pyridinium hydroxide, 4-vinyl-1-(3-sulphopropyl)pyridinium hydroxide, dimethyl(2-methacryloxyethyl)(3-sulphopropyl)ammonium hydroxide, diethyl(2-methacryloxyethoxy)-2-ethyl(3-sulphopropyl)-ammonium hydroxide, 4-vinyl-4-(sulphobutyl)pyridinium, N-(3-sulphopropyl)-N-methacrylamidopropyl-N,N-dimethylammonium hydroxide, N,N-dimethyl-N-(3-(methacrylamido)propyl)(3-sulphopropyl)ammonium hydroxide, N,N-dimethyl-N-(3-methacrylamidopropyl)-N-(3-carboxypropyl)ammonium hydroxide and N,N-dimethyl-N-(2-methacryloxyethyl)-N-(3-carboxypropyl)ammonium hydroxide.

7. Polymers according to any one of the preceding claims, characterized in that they also comprise units derived from nonionic ethylenic monomers.

8. Polymers according to claim 7, characterized in that the said nonionic ethylenic monomers are chosen from nonionic monomers that give, by polymerization, homopolymers with a glass transition temperature (Tg) of less than 0° C.

9. Polymers according to claim 7 or 8, characterized in that the nonionic ethylenic monomers are chosen from vinyl or allyl esters, (meth)acrylates and (meth)acrylamides of formula: 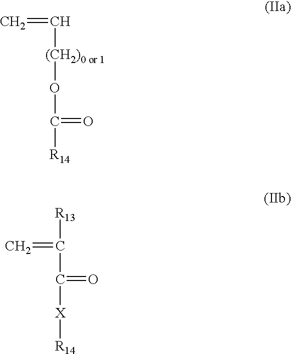
in which
R₁₃ represents a hydrogen atom or a methyl group,
X represents an oxygen atom or an NH group, and
R₁₄ represents a linear, branched, cycloaliphatic or aromatic C₂₋₆₀ hydrocarbon-based group, which may contain one or more atoms chosen from O, S and P, and especially an alkoxy-polyethylene glycol chain.

10. Polymers according to claim 9, characterized in that the nonionic ethylenic monomers are chosen from ethyl, n-butyl, n-hexyl, 2-ethylhexyl, n-nonyl, lauryl, n-octadecyl, isooctyl, isodecyl, hydroxyethyl, hydroxypropyl and methoxyethyl acrylate, n-hexyl, 2-ethylhexyl, ethoxyethyl, isodecyl, methoxyethyl or C₁₋₃₀alkoxy-PEG (with 5 to 30 ethylene oxide units) methacrylate, vinyl propionate, and vinyl neoalkanoates such as vinyl neononanoate and vinyl neododecanoate.

11. Polymers according to claim 10, characterized in that the nonionic ethylenic monomers are chosen from n-butyl acrylate, ethoxyethyl methacrylate, 2-ethylhexyl acrylate and methoxy-PEG methacrylate (with 5 to 30 ethylene oxide units).

12. Polymers according to claim 7, characterized in that the said nonionic ethylenic monomers are chosen from vinyl monomers containing a silicone side chain, chlorotrifluoroethylene, tetrafluoroethylene, and vinyl, allylic or (meth)acrylic monomers containing a perhalohydrocarbon and in particular a perfluorohydrocarbon side chain, such as perfluorohexyl (meth)acrylate or perfluorooctyl (meth)acrylate.

13. Cationic or amphoteric free-radical polymers according to any one of claims 8 to 12, characterized in that the units derived from cationic or amphoteric ethylenic monomers represent from 1% to 50% by weight of the polymer and preferably from 1% to 20% by weight of the polymer, and the units derived from nonionic ethylenic monomers giving homopolymers with a Tg<0° C. represent from 50% to 99% by weight of the polymer and preferably from 80% to 99% by weight of the polymer.

14. Polymers according to any one of the preceding claims, characterized in that they also comprise units derived from anionic ethylenic monomers.

15. Polymers according to claim 14, characterized in that the said anionic ethylenic monomers are chosen from acrylic acid, methacrylic acid, itaconic acid, crotonic acid, fumaric acid, maleic acid, vinylbenzoic acid, vinylbenzenesulphonic acid, acrylamidopropanesulphonic acid and vinylphosphonic acid, or from the addition salts with mineral or organic bases of these acids.

16. Polymers according to any one of the preceding claims, characterized in that they are chosen from:
copolymers of butyl acrylate and of dimethylaminoethyl methacrylate,
copolymers of butyl acrylate and of dimethylaminopropylmethacrylamide,
terpolymers of methoxy-PEG methacrylate (with 5 to 30 EO units), of dimethylaminopropylmethacrylamide and of ethoxyethyl methacrylate, and
terpolymers of 2-ethylhexyl acrylate, of dimethylaminopropylmethacrylamide and of ethoxyethyl methacrylate.

17. Cosmetic composition containing, in a cosmetically acceptable medium, at least one self-adhesive cationic or amphoteric polymer according to any one of the preceding claims.

18. Cosmetic composition according to claim 17, characterized in that it contains from 0.01% to 40%, preferably from 0.05% to 20% and in particular from 0.1% to 10% by weight, of at least one self-adhesive cationic or amphoteric polymer.

19. Cosmetic composition according to claim 17 or 18, characterized in that it is a care, makeup or fixing composition for human keratin materials, in particular for the hair and the eyelashes.

20. Cosmetic composition according to claim 19, characterized in that it is a styling composition.

21. Cosmetic composition according to claim 20, characterized in that it is a rinse-out styling composition.

22. Cosmetic composition according to one of claims 17 to 21, characterized in that it also contains additives chosen from surfactants, anionic, amphoteric, zwitterionic or nonionic polymers, cationic polymers other than the cationic self-adhesive polymers according to one of claims 1 to 16, nacreous agents and/or opacifiers, organic solvents, fragrances, mineral, plant and/or synthetic oils, fatty acid esters, pigments and colorants, silicones, mineral or organic particles, pH stabilizers, preserving agents and UV absorbers.

23. Cosmetic composition according to claim 22, characterized in that the cationic polymers are chosen from homopolymers of diallyldimethylammonium salt and copolymers of diallyldimethylammonium salt and of acrylamide.

24. Cosmetic composition according to claim 22, characterized in that the surfactants are chosen from anionic, nonionic, amphoteric and cationic surfactants, and mixtures thereof.

25. Cosmetic composition according to claim 24, characterized in that the anionic surfactants are present in a proportion of 0.5% to 60% by weight and preferably from 5% to 20% by weight, and in that the nonionic, amphoteric and cationic surfactants are present in a proportion of from 0.1% to 30% by weight and preferably from 0.5% to 25% by weight, relative to the total weight of the composition.

26. Cosmetic composition according to claim 22, characterized in that the silicones are chosen from volatile or non-volatile, cyclic, linear or branched silicones, optionally modified with organic groups.

27. Cosmetic composition according to claim 26, characterized in that the silicones are present in a proportion of from 0.01% to 20% by weight and preferably from 0.1% to 5% by weight.

28. Cosmetic use of the self-adhesive cationic or amphoteric free-radical polymers according to any one of claims 1 to 16.

29. Process for treating keratin materials, comprising the application of a cosmetic composition according to any one of claims 17 to 27 to the keratin materials to be treated.

30. Styling process comprising the application of a cosmetic composition according to any one of claims 17 to 27 to the hair, rinsing the hair, and then shaping and drying the rinsed hair.
